(19) United States
(12) Patent Application Publication
(10) Pub. No.: US 2023/0359953 A1
(43) Pub. Date: Nov. 9, 2023

(54) TASK ASSIGNMENT IN CLINICAL CARE ENVIRONMENT

(71) Applicant: Hill-Rom Services, Inc., Batesville, IN (US)

(72) Inventors: Neal Wiggermann, Batesville, IN (US); Craig M. Meyerson, Syracuse, NY (US)

(21) Appl. No.: 18/300,146

(22) Filed: Apr. 13, 2023

Related U.S. Application Data
(60) Provisional application No. 63/364,273, filed on May 6, 2022.

(51) Int. Cl. G06Q 10/0631; G16H 40/20; G06Q 10/0639

(52) U.S. Cl. CPC G06Q 10/063112; G06Q 10/0639; G16H 40/20

(57) ABSTRACT

A system for assigning nursing tasks determines a staff shortage in a clinical care environment exists. The system categorizes tasks based on skill level. The tasks are categorized into a first group of tasks for completion by a first type of personnel, and into a second group of tasks for completion by a second type of personnel. The second type of personnel have a skill set different from that of the first type of personnel. The system routes a task of the second group to a member of the second type of personnel.

### BACKGROUND

Healthcare facilities such as hospitals are presently experiencing nursing staff shortages. Temporary solutions for covering these shortages have included incentivizing overtime, offering hiring bonuses, and bringing in traveling nurses. However, these temporary solutions do not address the core concerns of nurses who are presently caring for more patients who are often sicker and require more specialized care than before. This has led to increasing burnout and departures from the nursing profession, which in turn has led to further nursing staff shortages. These shortages can negatively affect patient experience and outcomes.

### SUMMARY

In general terms, the present disclosure relates to assigning tasks in a clinical care environment. In one possible configuration, a system provides a technical effect by determining whether a staff shortage in a clinical care environment exists, and if so, routing tasks to an optimal volunteer based on consideration of one or more factors. Various aspects are described in this disclosure, which include, but are not limited to, the following aspects.

One aspect relates to a system for assigning nursing tasks, the system comprising: at least one processing device; and at least one memory storage device storing instructions which, when executed by the at least one processing device, cause the at least one processing device to: determine a staff shortage in a clinical care environment exists; categorize tasks based on skill level, the tasks being categorized into a first group of tasks for completion by a first type of personnel, and into a second group of tasks for completion by a second type of personnel, the second type of personnel having a skill set different from that of the first type of personnel; and route a task of the second group to a member of the second type of personnel.

Another aspect relates to a method of assigning nursing tasks, the method comprising: determining a staff shortage in a clinical care environment exists; categorizing tasks based on skill level, the tasks being categorized into a first group of tasks for completion by a first type of personnel, and into a second group of tasks for completion by a second type of personnel, the second type of personnel having a skill set different from that of the first type of personnel; and routing a task of the second group to a member of the second type of personnel.

Another aspect relates to a non-transitory computer readable storage medium, comprising instructions stored thereon which, when read and executed by one or more computing devices, cause the one or more computing devices to: determine a staff shortage in a clinical care environment exists; categorize tasks based on skill level, the tasks being categorized into a first group of tasks for completion by a first type of personnel, and into a second group of tasks for completion by a second type of personnel, the second type of personnel having a skill set different from that of the first type of personnel; and route a task of the second group to a member of the second type of personnel.

### DESCRIPTION OF THE FIGURES

The following drawing figures, which form a part of this application, are illustrative of the described technology and are not meant to limit the scope of the disclosure in any manner.

FIG. 1 schematically illustrates an example of a system that includes a task assignment server for assigning tasks in a clinical care environment.

FIG. 2 schematically illustrates an example of a method of assigning tasks that can be performed by the task assignment server of FIG. 1.

FIG. 3 illustrates an example of a device displaying a task request that can be generated by the task assignment server of FIG. 1.

FIG. 4 illustrates an example of a device displaying a task list that can be generated by the task assignment server of FIG. 1.

FIG. 5 schematically illustrates an example of a method of monitoring performance of tasks that can be generated by the task assignment server of FIG. 1.

FIG. 6 illustrates an example of a volunteer history screen that can be generated by the task assignment server of FIG. 1 for display on a device belonging to a volunteer in the clinical care environment.

FIG. 7 illustrates an example of an achievements and rewards screen that can be generated by the task assignment server of FIG. 1 for display on a device belonging to a volunteer in the clinical care environment.

FIG. 8 illustrates an example of a task acceptance screen that can be generated by the task assignment server of FIG. 1 for display on a device belonging to a volunteer in the clinical care environment.

FIG. 9 illustrates an example of a task assignment screen that can be generated by the task assignment server of FIG. 1 for display on a device belonging to a volunteer in the clinical care environment.

FIG. 10 illustrates an example of an assign task screen that can be generated by the task assignment server of FIG. 1 for display on a device of a nursing staff member in the clinical care environment.

FIG. 11 illustrates an example of a task assignment request screen that can be generated by the task assignment server of FIG. 1 for display on a device of a nursing staff member in the clinical care environment.

FIG. 12 schematically illustrates an example of the task assignment server of FIG. 1.

### DETAILED DESCRIPTION

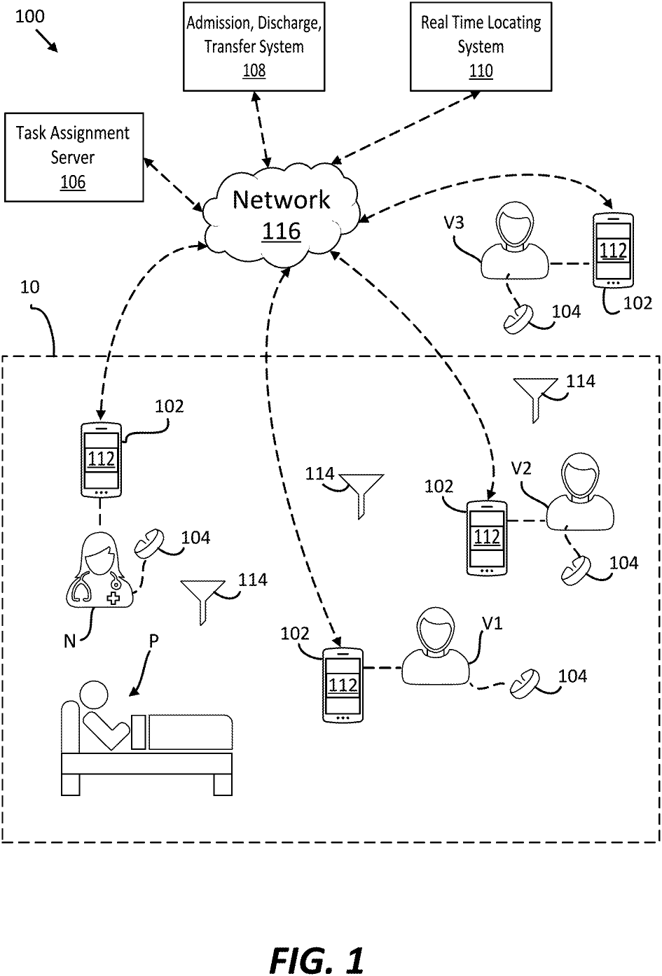

FIG. 1 schematically illustrates an example of a system 100 for assigning nursing tasks. As shown in FIG. 1, a nursing staff member N provides care to a patient P inside a clinical care environment 10. The clinical care environment 10 can include a hospital, a nursing home, a long-term care facility, and other types of environments for providing clinical care.

As shown in FIG. 1, volunteers V are located inside and outside of the clinical care environment 10. The volunteers V can include nursing students, idle family members of patients admitted to the clinical care environment 10, administrative staff and other employees of the clinical care environment 10, freelance laborers located outside of the clinical care environment 10, and other types of individuals to whom tasks can be assigned for completion inside the clinical care environment 10. In some examples, the nursing staff members N are classified as a first type of personnel, and the volunteers V are classified as a second type of personnel who have a different skill set and/or clinical experience than the first type of personnel.

Each of the nursing staff members N and the volunteers V operates a device 102 on which a task assignment application 112 is installed or is accessible. Examples of the devices 102 can include smartphones, tablet computers, or other type of computing devices providing access to the task assignment application 112. In some examples, the task assignment application 112 can be downloaded on the devices 102. In further examples, the task assignment application 112 can be a web-based or cloud-based application that is accessible on the devices 102. As an example, the task assignment application 112 can be hosted or otherwise supported by a task assignment server 106, which will be described in more detail below.

In certain examples, the task assignment server 106 can provide communication channels allowing the nursing staff members N, administrators, or patients P to leave video, audio, or text messages to thank the volunteers V based on performance of their assigned tasks. In some examples, the task assignment application 112 is an extension of the Voalte® platform available from Hillrom Holdings, Inc. In certain examples, the task assignment application 112 incorporates elements of the systems and methods described in U.S. Pat. No. 9,749,825, granted Aug. 29, 2017, titled Connection-Oriented Messaging and Signaling in Mobile Health Networks, and U.S. Pat. No. 9,872,148, granted Jan. 16, 2018, titled Adaptive Mobile Wireless Call Rescue, which are incorporated herein in their entireties.

The task assignment application 112 enables the nursing staff member N to assign tasks to the volunteers V when the nursing staff member N is unable or too busy to perform the tasks such as during a staff shortage at the clinical care environment 10. Staff shortages can occur due to a medical surge when a sudden increase in patient admissions to the clinical care environment 10 challenges or exceeds the care capacity of the clinical care environment 10.

The assignment of tasks from the nursing staff member N to the volunteers V is managed by the task assignment server 106 across a communications network 116. As shown in FIG. 1, the devices 102 used by the nursing staff member N and the volunteers V are all connected to the communications network 116. The communications network 116 can include any type of wired or wireless connections or any combinations thereof. Examples of wireless connections include Wi-Fi, Bluetooth, and broadband cellular networks including 4G or 5G. In some examples, the communications network 116 includes a broadband cellular network. In some examples the communications network 116 includes the Internet.

In one embodiment, the nursing staff member N can manually request help for certain tasks and patients. In such examples, a task assignment request from the nursing staff member N is received by the task assignment server 106 through a connection with the device 102 operated by the nursing staff member N via the communications network 116. The task assignment server 106 can then route the task assignment request to a volunteer V using routing logic based on at least one of an availability of the volunteer, a skill level of the volunteer, a physical ability of the volunteer, one or more preferences of the volunteer, and a location of the volunteer relative to a location where the task is to be performed in the clinical care environment 10.

In another embodiment, the task assignment server 106 can proactively assign or triage tasks automatically without requiring manual input from the nursing staff member N. For example, the task assignment server 106 can determine a staff shortage exists in the clinical care environment, and then automatically route task assignment requests to the volunteers V using routing logic based on at least one of availability of the volunteers, skill level of the volunteers, physical ability of the volunteers, preferences of the volunteers, and locations of the volunteers relative to the locations where the tasks are to be performed in the clinical care environment 10.

As a further illustrative example, the task assignment server 106 can consider the tasks that are accepted and in queue for completion by a volunteer V, and can estimate the completion time for each task in the queue based on the type of task, the volunteer V’s distance from the patient P, patient P’s location as it relates to the task (e.g., distance from ice machine), and other types of factors. The task assignment server 106 can then determine the volunteer V’s availability based on the estimated completion time of the tasks accepted by the volunteer V, and can route new task assignment requests to the volunteer V based on their availability. As another example, the task assignment server 106 can optimize the assignment of tasks such as by assigning low-skilled tasks to lower skilled volunteers to leave open availability of higher skilled volunteers. Such optimization can especially occur when the task assignment server 106 determines that higher skilled tasks are likely to be assigned based on historical data and trends.

The routing logic that is used by the task assignment server 106 to route the task assignment requests to the volunteers V can be customized. For example, the routing logic can be customized based on the individual needs of the nursing staff member N, the needs of the team, unit, or department within the clinical care environment 10 where the nursing staff member N is assigned their shift, or the overall needs of the clinical care environment 10.

Examples of the tasks that can be assigned by the task assignment server 106 to the volunteers V can include, without limitation, getting ice chips and water, feeding the patient P, helping the patient P to the bathroom or to ambulate around the clinical care environment 10, holding and/or calming the patient P during a medical procedure, watching the patient P to prevent falls (e.g., being a sitter), calming the patient P, preventing extubation of an endotracheal tube (ET), monitoring the patient P for seizures, accompanying the patient P for end of life (e.g., hospice care), and other common types of tasks for providing care in the clinical care environment 10. In accordance with the foregoing examples, the tasks assigned by the task assignment server 106 to the volunteers V often do not require advanced medical training.

The task assignment server 106 can check one or more credentials of the volunteers V to ensure that the tasks are only assigned to volunteers V with appropriate experience, training, and/or physical capabilities. For example, tasks such as retrieving ice chips, feeding a patient, or accompanying a lonely patient may be open to all volunteers V, but a task of helping a patient to the bathroom may only be assigned to volunteers V with basic training. The volunteers V may also set the tasks that they prefer doing or are unable/unwilling to do which would be considered by the task assignment server 106 when assigning the tasks to the volunteers V.

In some examples, the task assignment server 106 can be programmed to perform advance scheduling for some tasks. For example, a volunteer V may be scheduled to help feed patients at lunchtime, or if a patient needs a “sitter” to prevent falls, shifts can be scheduled in advance. Administrators may also add tasks that are less time sensitive (e.g., replacing the batteries in clocks) to a queue that could be assigned after more urgent tasks are completed.

Advantages of the task assignment server 106 for the patient P can include eliminating the need to transfer the patient P to another clinical care environment or location that has care capacity, and minimizing patient deterioration through faster clinical intervention. Advantages of the task assignment server 106 for the nursing staff member N can include receiving assistance during medical surges to reduce fatigue and burnout. Further advantages of the task assignment server 106 can include increasing the care capacity of the clinical care environment 10 to provide clinical care for a higher number of admitted patients.

As shown in FIG. 1, an admission, discharge, and transfer (ADT) system 108 is connected to the communications network 116. The ADT system 108 tracks patients from their moment of arrival at the clinical care environment 10 until their departure, and can store relevant patient information such as medical record numbers, names, and contact information. As will be described in more detail below, the ADT system 108 can be used to determine whether a staff shortage exists in the clinical care environment 10 based on patient volume.

As further shown in the example provided in FIG. 1, the nursing staff member N and the volunteers V each wear or otherwise carry a tag 104 that is detectable by antennas 114 positioned throughout the clinical care environment 10. The antennas 114 are fixed reference points that receive wireless signals from the tags 104. The antennas 114 communicate the wireless signals from the tags 104 to a real-time locating system (RTLS) 110 via the communications network 116. The RTLS 110 uses the data acquired from the antennas 114 to monitor and track the location of the tags 104 (and of the object to which they are attached such as the nursing staff member N and the volunteers V) inside the clinical care environment 10.

In alternative examples, the location of the nursing staff member N and the volunteers V can be monitored by tracking the movement of the devices 102. In examples where the devices 102 are portable computing devices such as smartphones or table computers that are carried by the nursing staff member N and the volunteers V, the location of the devices 102 can be tracked by various tracking techniques including multilateration of radio signals between cell towers of a telecommunications network and the devices 102, or by using geo-spatial positioning techniques by satellite navigation systems such as the Global Positioning System (GPS).

As shown in the example of FIG. 1, a first volunteer V1 and a second volunteer V2 are both located inside the clinical care environment 10, while a third volunteer V3 is located outside of the clinical care environment 10. In this example, the first volunteer V1 is shown as being physically located in closer proximity to the patient P than the second and third volunteers V2, V3. In this example, the routing logic used by the task assignment server 106 can factor in the relative location of the first, second, and third volunteers V1, V2, V3 with respect to the location where the task is to be performed (which will often be where the patient P is located) when determining which volunteer V to route a task assignment request to.

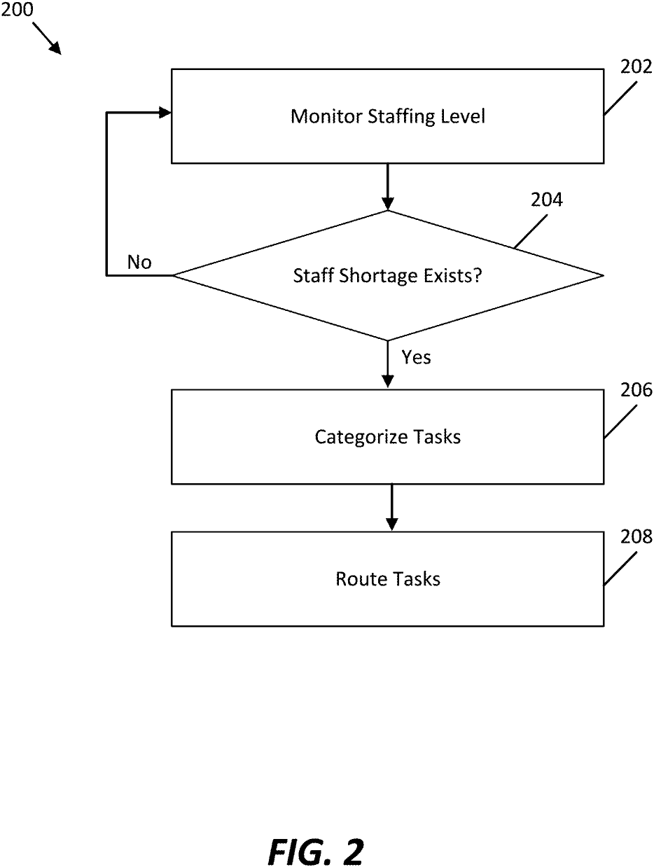

FIG. 2 schematically illustrates an example of a method 200 of assigning nursing tasks in the clinical care environment 10. In some instances, the method 200 is performed by the task assignment server 106. The method 200 can be performed to optimize resources across units of the clinical care environment 10, or to take specific actions when extreme workforce pressures are present. For example, the method 200 can be used to direct resources from one or more units to a unit in need. These resources can include the volunteers V, low-skilled floating employees of the clinical care environment 10 such as environmental services (EVS) or transporters, and/or high-skilled floating employees and administrative staff of the clinical care environment 10.

The method 200 includes an operation 202 of monitoring a staffing level of a first type of personnel in the clinical care environment 10. In some examples, the first type of personnel includes the nursing staff members N. In further examples, the first type of personnel includes additional types of medical professionals in the clinical care environment 10.

The staffing level of the first type of personnel can be monitored by tracking how many of the first type of personnel (e.g., nursing staff members N) are logged into the task assignment server 106. Alternatively, the staffing level of the first type of personnel can be monitored by tracking a presence and/or location of the first type of personnel (e.g., nursing staff members N in the clinical care environment 10 using the RTLS 110. As described above, the RTLS 110 receives data from the antennas 114 that are placed throughout the clinical care environment 10. The antennas 114 are used to detect the presence and/or location of the tags 104 worn by the first type of personnel (e.g., nursing staff members N).

Next, the method 200 includes an operation 204 of determining whether a staffing shortage exists in the clinical care environment 10. In one example, the staff shortage is determined by receiving a trigger input from a manager of the first type of personnel. For example, a staffing shortage can be triggered by a nurse manager responsible for managing the nursing staff members N in the clinical care environment 10. In this example, the staffing shortage is manually triggered. Advantageously, the manager can use their firsthand observations of the clinical care environment 10 to determine whether to trigger a staff shortage.

In another example, the staff shortage is determined when average wait times for completion of tasks by the first type of personnel (e.g., nursing staff members N) exceed a threshold set for the clinical care environment 10. In this example, the staffing shortage is automatically triggered by the task assignment server 106. For example, the task assignment server 106 can monitor average times for completion of tasks by monitoring usage of the task assignment application 112 by the nursing staff members N. The threshold can be set by a manager or administrator in the clinical care environment 10, and the average wait times can be compared to the threshold for determining whether to trigger the staff shortage in operation 204.

In another example, the staff shortage is determined when a ratio of the first type of personnel (e.g., nursing staff members N) to patients exceeds a threshold set for the clinical care environment. This is another example of when the staffing shortage is automatically triggered by the task assignment server 106. In one example, the ratio of the first type of personnel to patients is calculated by determining a total number of the first type of personnel logged into the task assignment application 112, determining a total number of patients admitted to the clinical care environment 10 using data acquired from the ADT system 108, and then comparing the total number of the first type of personnel to the total number of patients.

In another example, the ratio of the first type of personnel to patients is calculated by determining a total number of the first type of personnel in the clinical care environment using location data acquired from the RTLS 110, determining a total number of patients admitted to the clinical care environment 10 using data acquired from the ADT system 108, and then comparing the total number of the first type of personnel to the total number of patients.

As shown in FIG. 2, when a staff shortage is determined not to exist (i.e., “No” in operation 204), the method 200 returns to operation 202 of monitoring the staffing level of the first type of personnel in the clinical care environment 10. When a staff shortage is determined to exist (i.e., “Yes” in operation 204), the method 200 proceeds to operation 206 of categorizing tasks into categories for completion by different personnel in the clinical care environment 10.

In one example, operation 206 includes categorizing the tasks based on skill level. For example, operation 206 can include categorizing the tasks into a first group of tasks for completion by the first type of personnel (e.g., the nursing staff members N), and into a second group of tasks for completion by a second type of personnel (e.g., the volunteers V). In this example, the second type of personnel have a different skill set than the first type of personnel. For example, the volunteers V can have a lower skill level the nursing staff members N.

Next, the method 200 includes an operation 208 of routing tasks in the second group of tasks to members of the second type of personnel (e.g., the volunteers V). In some examples, the task assignment server 106 routes the tasks in the second group of tasks to the devices 102 of the members of the second type of personnel using the communications network 116.

The task is routed to the members of the second type of personnel based on routing logic that factors at least one of availability of the members (including their availability based on estimated completion time of accepted tasks in queue for completion by each member of the second type of personnel), skill level of the members, physical ability of the members, preferences of the members, and proximity of the members to locations where tasks are scheduled for completion in the clinical care environment 10. This ensure the tasks are routed to the most qualified or well-suited member of the second type of personnel.

As an illustrative example, when a task is related to providing care to the patient P shown in FIG. 1, operation 208 can include routing the task to the first volunteer V1 who is in closer proximity to the patient P than the second and third volunteers V2, V3, when the first volunteer V1 is available, and has an appropriate skill level and physical ability to complete the task. The relative distances between the second type of personnel (e.g., the volunteers V) and the locations for performing tasks (e.g., the patient P) can be determined by using data acquired from the RTLS 110 that tracks the locations of the volunteers V, the patients P, the nursing staff members N, and other personnel and objects in the clinical care environment 10.

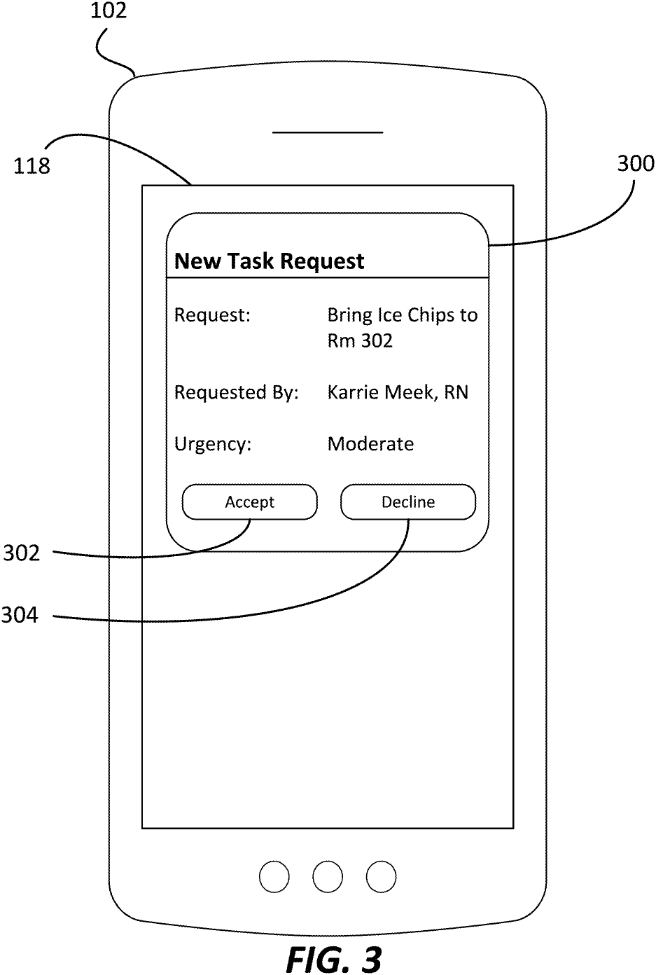

FIG. 3 illustrates an example of a device 102 displaying a task request 300 generated by the task assignment server 106, and routed to the device 102 which belongs to a volunteer V, in accordance with the examples described above. In this example, the task request 300 is displayed on a display 118 of the device 102. In some examples, the display 118 is a touchscreen that both displays outputs (e.g., the task request 300) and receives tactile inputs from the volunteer V. In this example, the task request 300 includes information that identifies the task (e.g., “Bring Ice Chips to Rm 302”), the person who made the request (e.g., “Karrie Meek, RN”), and the urgency level for completing the task (e.g., “Moderate”). It is contemplated that the task request 300 may include additional information or less information, which can be customized based on the needs of the clinical care environment 10.

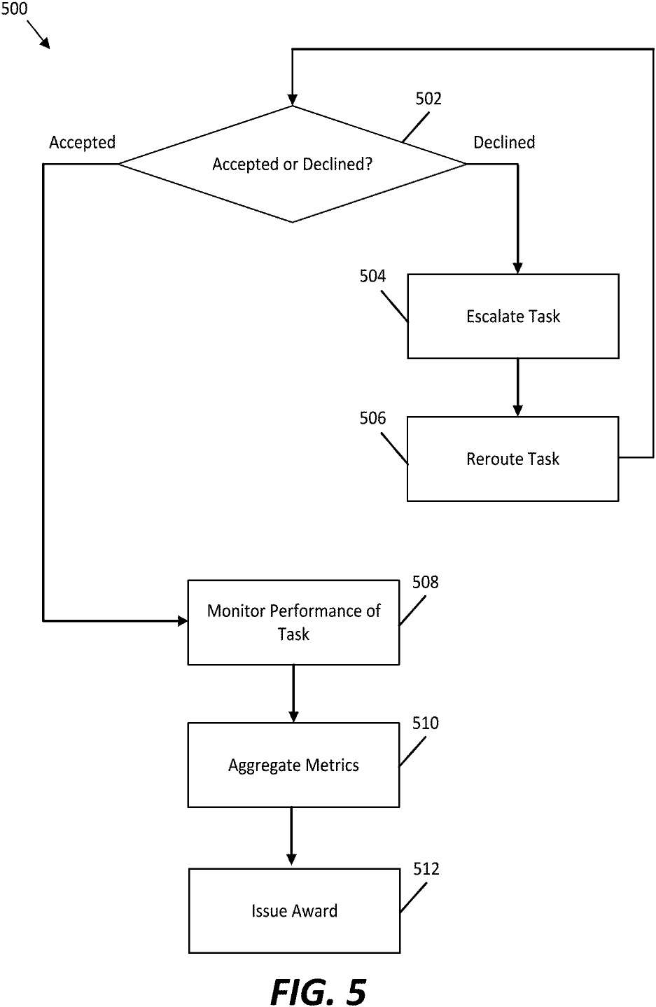

In the example shown in FIG. 3, the task request 300 provides an option 302 for the volunteer V to accept the task request 300, and an option 304 for the volunteer V to decline the task request 300. When the volunteer V selects the option 302 to accept the task request 300, the task assignment server 106 assigns the task request 300 to the volunteer V for completion. When the volunteer V selects the option 304 to decline the task request 300, the task assignment server 106 escalates and reassigns the task request to another volunteer V. These steps will be described in more detail below with reference to the method 500 schematically illustrated in FIG. 5. In alternative examples, the task request 300 does not provide the options 302, 304, and instead the task request 300 is automatically assigned to the volunteer V without first requiring their acceptance. In such instances, a notification can be simply displayed on the device 102 of the volunteer V that notifies them that they have been assigned a new task for completion.

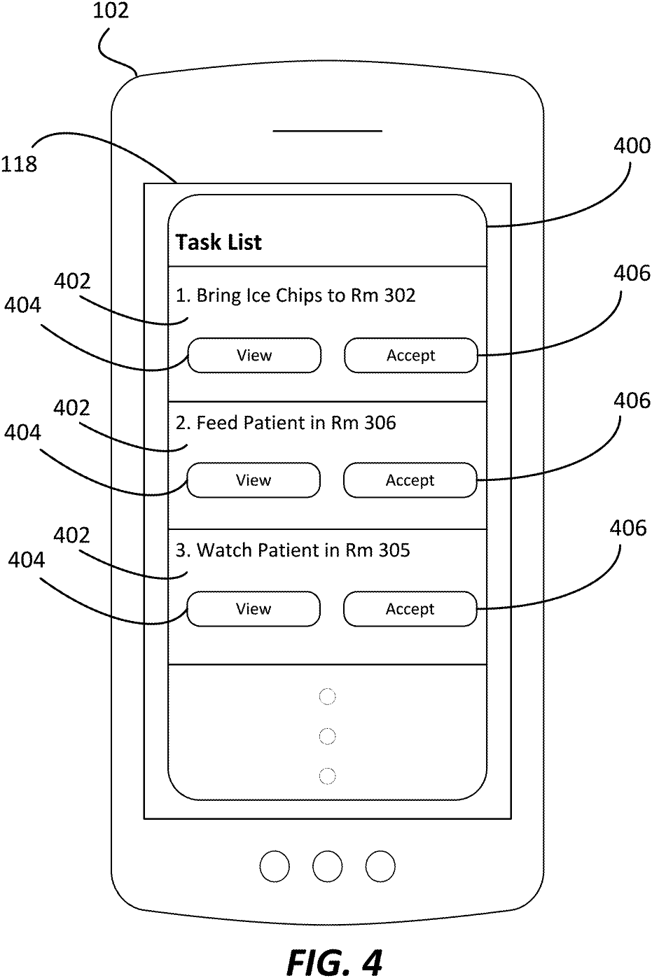

FIG. 4 illustrates an example of a device 102 displaying a task list 400 generated by the task assignment server 106. In this example, the task list 400 is displayed on the display 118 of the device 102, which belongs to a volunteer V. The task list 400 includes one or more task requests 402 that have been routed to the volunteer V in accordance with the examples described above. Each of the task requests 402 includes an option 404 for the volunteer V to view additional information of the task request 402 (e.g., to view information included in the task request 300 of FIG. 3), and an option 406 for the volunteer V to accept the task request 402.

In this example, the task requests 402 that are included in the task list 400 are routed to the volunteer based on the availability of the volunteer, skill level of the volunteer, physical ability of the volunteer, preferences of the volunteer, and location of the volunteer such that the task list 400 includes task requests 402 that are well-suited for completion by the volunteer. The task list 400 can be dynamically updated based on acceptance and/or completion of the task requests 402 by the volunteer, and by other volunteers in the clinical care environment 10.

Also, the order of the task requests 402 in the task list 400 can be dynamically updated based on escalation of the task requests 402. For example, when a task request 402 remains unassigned or uncompleted for more than a predefined amount of time, the task request 402 is escalated to have a higher urgency level, which can cause the task request 402 to have a higher ranking in the task list 400, or to have a different appearance such as a different color, font size, or other visual indicator to communicate the higher urgency level. Accordingly, the task list 400 can dynamically rank the task requests 402 based on urgency such that task requests 402 having a higher urgency are ranked before task requests 402 having a lower urgency.

FIG. 5 schematically illustrates an example of a method 500 of monitoring performance of tasks by the volunteers V in the clinical care environment 10. The method 500 can be performed by the task assignment server 106 after the tasks are routed to the volunteers V in operation 208 of the method 200 shown in FIG. 2.

As shown in FIG. 5, the method 500 includes an operation 502 of determining whether a task request is accepted or declined by a volunteer V. When the task request is declined, the method 500 can include an operation 504 of escalating the task request to have a higher priority, followed by an operation 506 of rerouting the task request to another volunteer V in the clinical care environment. Thereafter, the method 500 can return to operation 502 for determining whether the task request is accepted or declined by the other volunteer V. In certain examples, each time a task request is declined by a volunteer, the task request is escalated to have a higher priority. In further examples, the task request is escalated to have a higher priority based on other metrics such as a time duration for the task request to accepted or completed. In some scenarios where no volunteer V accepts the task request, the task assignment server 106 notifies the nursing staff member N that the task request has not been accepted. Alternatively, the task assignment server 106 can provide a screen on the device 102 of the nursing staff member N allowing the nursing staff member N to check the status of their open task requests.

When operation 502 determines that the task request is accepted, the method 500 proceeds to an operation 508 of monitoring performance of the task. Operation 508 can include monitoring metrics such as whether the task is completed or not. When the task is determined as completed, operation 508 can further include monitoring additional metrics such as the time it took to complete the task, and satisfaction by the patient P and/or nursing staff member N.

Next, the method 500 can include an operation 510 of aggregating the metrics monitored in operation 508 with metrics from additional tasks completed by the volunteers. For example, operation 510 can include calculating a total number of tasks completed by a volunteer V over a period of time such as a day, a week, or a month. Operation 510 can include calculating metric averages such as average time for completion of each task, average satisfaction of the patients P and nursing staff members N for the tasks completed by the volunteer.

Next, the method 500 includes an operation 512 of issuing an award to one or more of the volunteers V based on the metrics aggregated in operation 510. For example, an award can be an achievement or a reward that is issued to a volunteer V who completed the highest total number of tasks or who performed the longest cumulative amount of time for tasks over a predetermined period of time such as a day, a week, or a month. As another example, an award can be an achievement or a reward that is issued to a volunteer V who had the highest satisfaction rate among the patients P and/or the nursing staff members N in the clinical care environment 10. Additional awards based on additional types of metrics can also be issued.

The awards can provide encouragement and motivation to the volunteers V, which can increase engagement and attendance by the volunteers V, and/or provide acknowledgement to students and non-clinical staff who go beyond their standard responsibilities. Gamification can create goals and/or achievements for the volunteers V. In some instances, physical or virtual badges are awarded to volunteers V, which can be traded for items in a cafeteria or gift shop of the clinical care environment 10. A volunteer of the month or an “unit angel” can be designated based on certain performance metrics (e.g., tasks completed, time on unit, and the like).

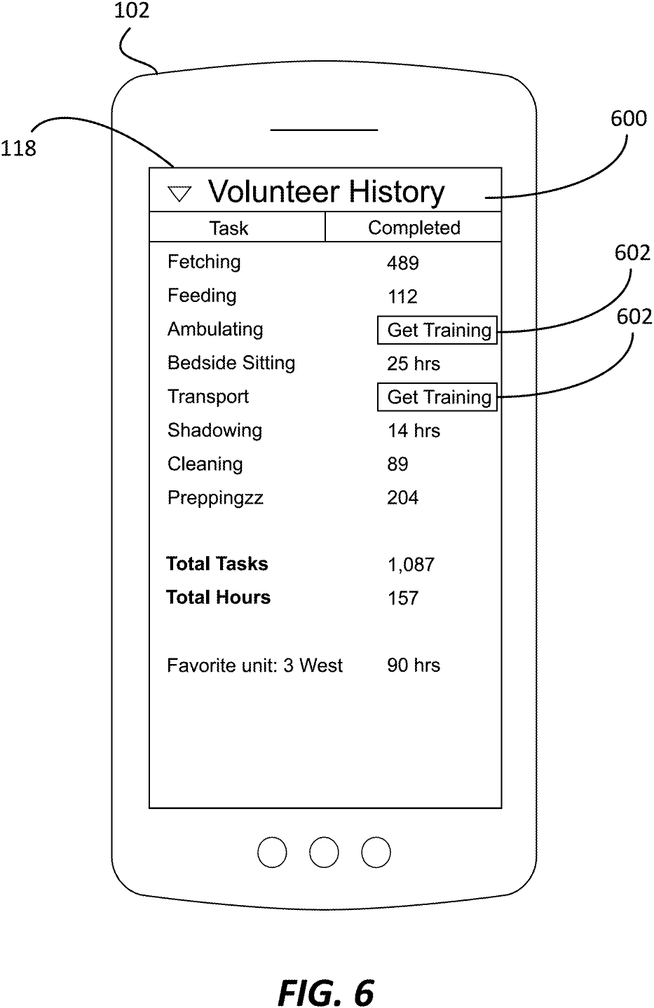

FIG. 6 illustrates an example of a volunteer history screen 600 that can be generated by the task assignment server 106 for display on a device 102 belonging to a volunteer V in the clinical care environment 10. The data displayed on the volunteer history screen 600 can include the metrics that are aggregated in operation 510 of the method 500. The volunteer history screen 600 can display a number of times a task was completed or a cumulative time duration for certain tasks. In the example shown in FIG. 6, the volunteer history screen 600 provides data indicating that a volunteer V completed fetching tasks 489 times, feeding tasks 112 times, cleaning tasks 89 times, and prepping tasks 204 times. The volunteer history screen 600 further shows that the volunteer performed bedside sitting for 25 cumulative hours and shadowing for 14 cumulative hours. For tasks that the volunteer V is not qualified to perform, the volunteer history screen 600 includes links 602 for the volunteer to select which can either cause a training video to be displayed on the display 118 of the device 102, or schedule a nursing staff member N to meet with the volunteer V to provide them the appropriate training.

As further shown in the example provided in FIG. 6, the volunteer history screen 600 can provide a summary of a total number of tasks completed (e.g., 1,087) and/or a total number of hours performed for tasks measured by time duration (e.g., sitting and shadowing events). The volunteer history screen 600 can further identify the unit or department within the clinical care environment where the volunteer performed most of their tasks or spent most of their time.

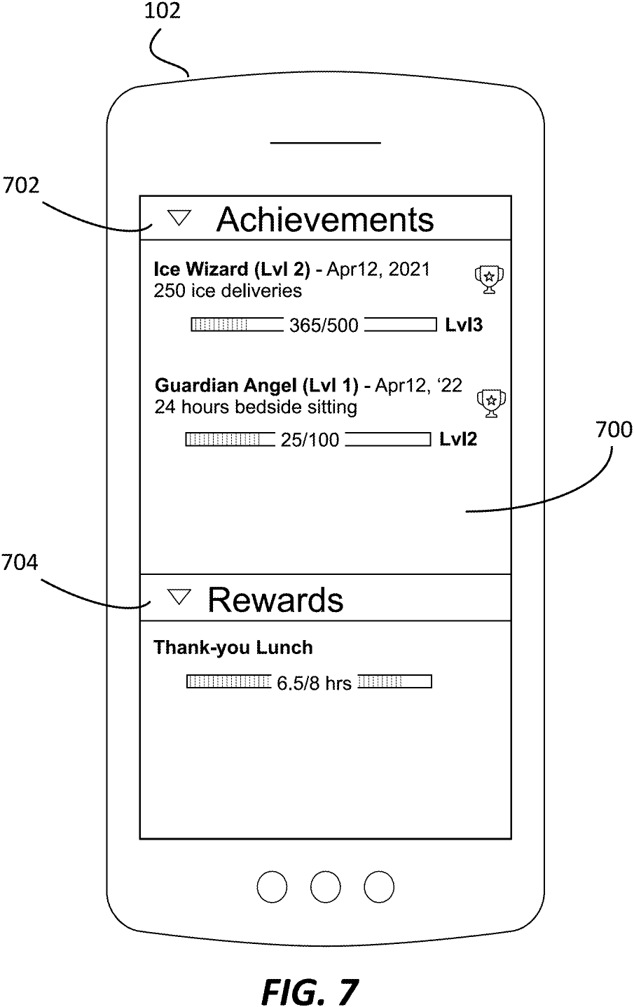

FIG. 7 illustrates an example of an achievements and rewards screen 700 that can be generated by the task assignment server 106 for display on a device 102 belonging to a volunteer V in the clinical care environment 10. The data displayed on the achievements and rewards screen 700 can include the awards issued in operation 512 of the method 500, which can be based on the aggregated metrics shown in the volunteer history screen 600 and aggregated in operation 510 of the method 500. As shown in FIG. 7, the achievements and rewards screen 700 can display achievements 702 (e.g., “Ice Wizard” or “Guardian Angel”) that each include a goal (e.g., 500 ice deliveries or 100 hours) and an aggregated number of times (e.g., 365 ice deliveries) or cumulative time duration that the volunteer V has completed (e.g., 25 hours) with respect to the goal. Thus, the volunteer V can know how many more times they need to complete a task or how much more time they need to perform to satisfy the achievement. The achievements and rewards screen 700 can further display rewards 704 (e.g., Thank you lunch”) that can similarly include a goal (e.g., 8 hours) and an aggregated number of times or cumulative time duration that the volunteer V has completed (e.g., 6.5 hours) with respect to the goal.

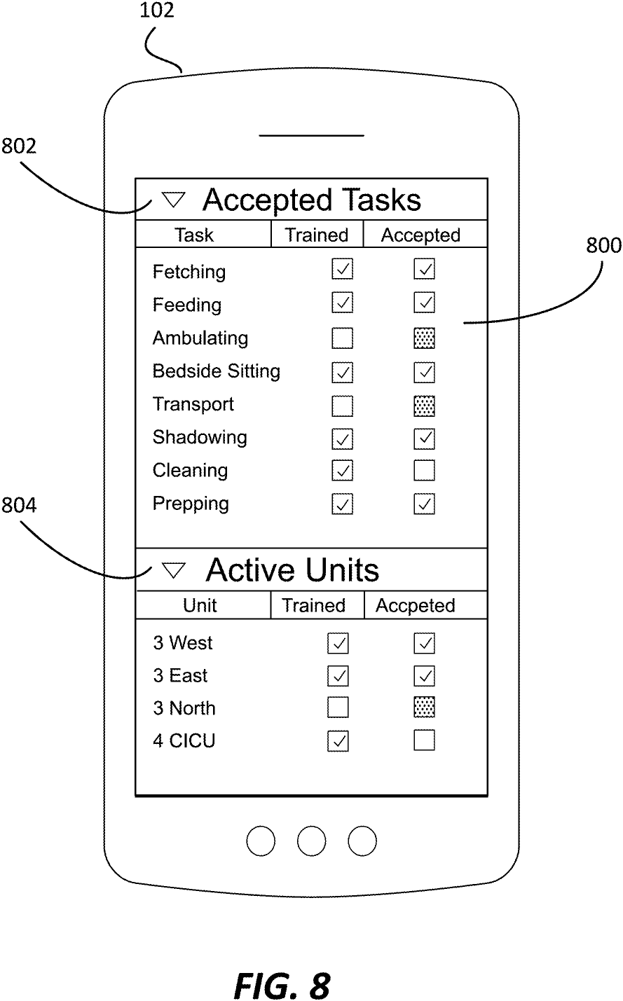

FIG. 8 illustrates an example of a task acceptance screen 800 that can be generated by the task assignment server 106 for display on a device 102 belonging to a volunteer V in the clinical care environment 10. The task acceptance screen 800 displays tasks 802 (e.g., fetching, feeding, ambulating, bedside sitting, transport, shadowing, cleaning, prepping, etc.). For each of the tasks 802, the task acceptance screen 800 identifies (1) whether the volunteer V is trained to perform the task and (2) whether the volunteer V accepts the type of task. The volunteer V can only accept tasks for which they have received training or are otherwise qualified to perform. A task for which the volunteer V is not trained (e.g., ambulating) can be greyed or blocked on the task acceptance screen 800, which prevents the volunteer V from accepting the task. Certain types of tasks require no training such that these tasks are automatically marked as trained. In this example, only the tasks 802 that the volunteer V accepts on the task acceptance screen 800 are routed to the device 102 by the task assignment server 106.

The task acceptance screen 800 further displays units or departments 804 within the clinical care environment 10 for which the volunteer V has accepted to receive the tasks 802 from. In this example, only tasks 802 from the units or departments 804 that the volunteer V accepts are routed to the device 102 by the task assignment server 106.

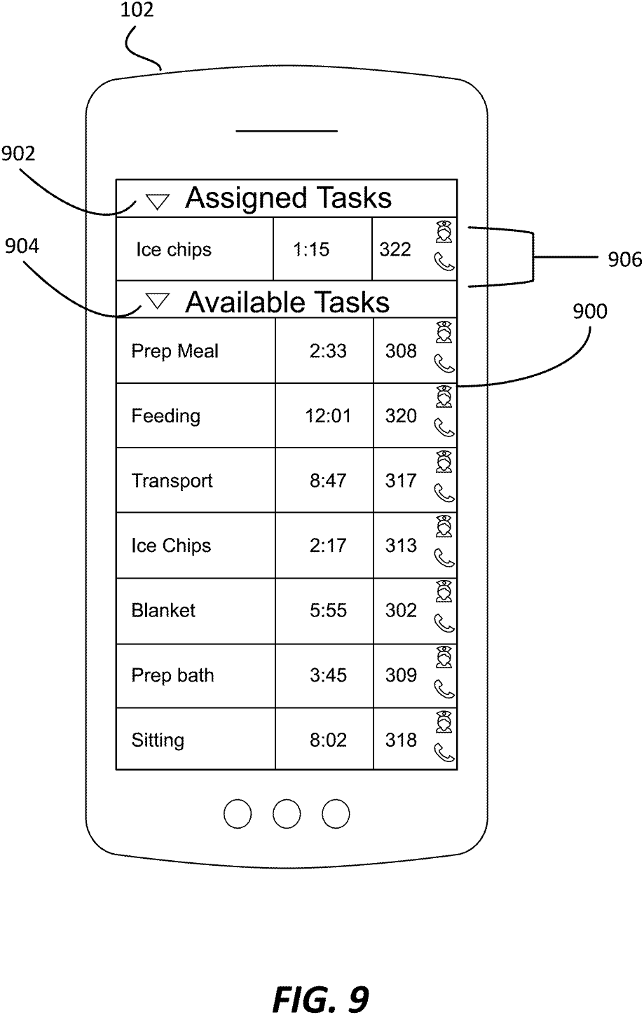

FIG. 9 illustrates an example of a task assignment screen 900 that can be generated by the task assignment server 106 for display on a device 102 belonging to a volunteer V in the clinical care environment 10. As shown in the example of FIG. 9, the task assignment screen 900 displays assigned tasks 902 that can include information that identifies the task (e.g., “Ice Chips”), the time for completing the task (e.g., “1:15”), and the location for completing the task (e.g., “Rm 322”). Each assigned task 902 can also include text and/or call icons 906 for the volunteer to text and/or call the nursing staff member N associated with the assigned task 902.

The task assignment screen 900 can further display available tasks 904 that have not yet been assigned. The available tasks 904 can include similar information as the assigned tasks 902 such as information that identifies the task, the time for completing the task, and the location for completing the task. Each of the available tasks 904 can be selected by the volunteer V such that the available tasks become assigned tasks 902 for completion by the volunteer V.

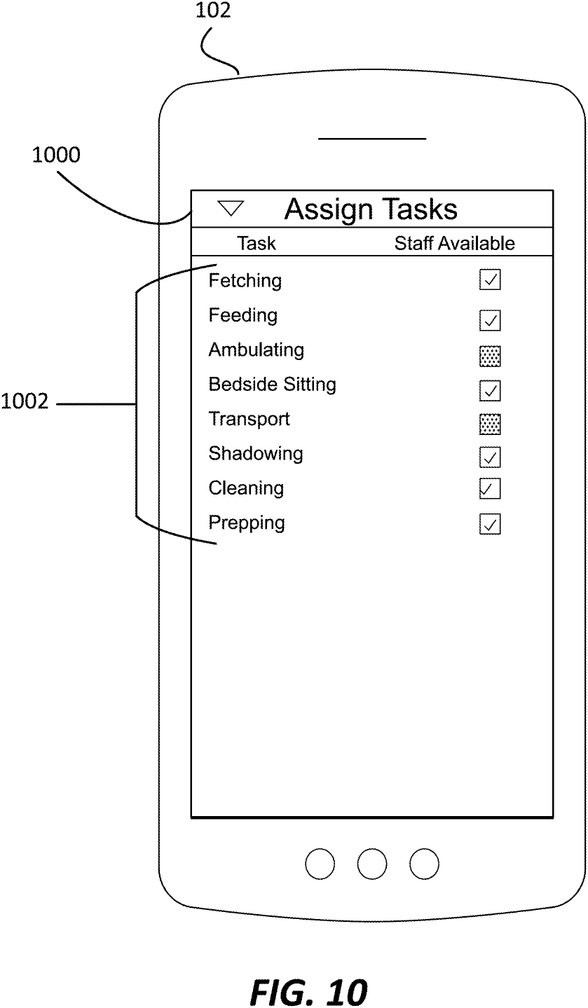

FIG. 10 illustrates an example of an assign task screen 1000 that can be generated by the task assignment server 106 for display on a device 102 of a nursing staff member N in the clinical care environment 10. The assign task screen 1000 includes tasks 1002 that can be selected by the nursing staff member N for assignment to the volunteers V. A task 1002 for which there are no available volunteers V (e.g., due to lack of training) can be greyed or blocked on the assign task screen 1000, which prevents the nursing staff member from assigning the task.

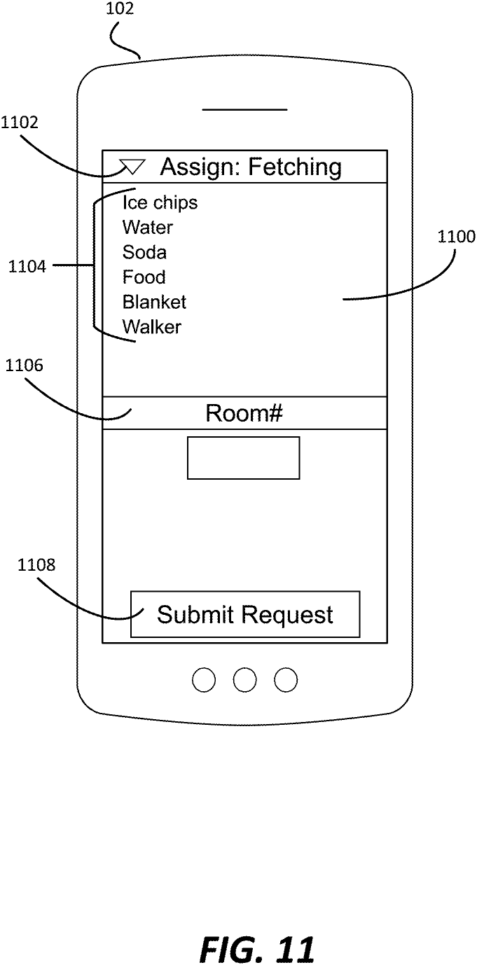

FIG. 11 illustrates an example of a task assignment request screen 1100 that can be generated by the task assignment server 106 for display on a device 102 of a nursing staff member N in the clinical care environment 10. In this example, a nursing staff member N can assign a task 1102 (e.g., “fetching”) for one or more items 1104 (e.g., “Ice Chips, “Water”., “Soda”, “Food”, “Blanket”, “Walker”, and the like) and a location 1106 for bring the items 1104 to such as a room number within the clinical care environment 10. In some examples, the location 1106 can include a current room or location input button that automatically tags the location of the nursing staff member N to the task 1102 such that nursing staff member N does not have to manually enter the location for completion of the task. Once the parameters for the task 1102 (e.g., the items 1104 and the location 1106) are entered, the nursing staff member N can select a submit icon 1108 to submit a request for assigning the task 1102 to a volunteer V.

In some further examples, the task assignment server 106 allows the nursing staff member N to verbally submit a request for assignment of a task 1102 to a volunteer V such as through a voice command when enabled on the device 102 of the nursing staff member N. Also, the device 102 of the volunteer V can similarly accept voice commands from the volunteer V that allow the volunteer V to verbally accept the task 1102 on their device 102.

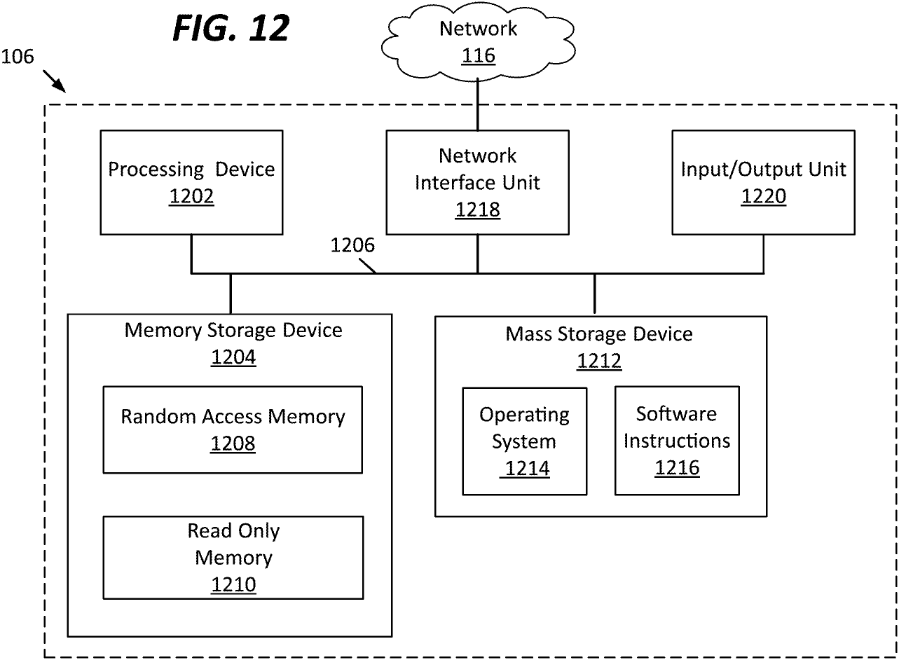

FIG. 12 schematically illustrates an example of the task assignment server 106 that can be used to implement aspects of the task assignment application 112. The task assignment server 106 includes a processing device 1202, a memory storage device 1204, and a system bus 1206 that couples the memory storage device 1204 to the processing device 1202. The processing device 1202 is an example of a central processing unit (CPU). The devices 102 that are operated by the nursing staff members N and the volunteers V in the clinical care environment 10 can include similar computing components.

As shown in FIG. 12, the memory storage device 1204 can include a random-access memory (“RAM”) 1208 and a read-only memory (“ROM”) 1210. Basic input and output logic having basic routines that help to transfer information between elements within the task assignment server 106, such as during startup, can be stored in the ROM 1210.

The task assignment server 106 can also include a mass storage device 1212 that can include an operating system 1214 and store software instructions 1216 and data. The mass storage device 1212 is connected to the processing device 1202 through the system bus 1206. The mass storage device 1212 and associated computer-readable data storage media provide non-volatile, non-transitory storage for the task assignment server 106.

Although the description of computer-readable data storage media contained herein refers to the mass storage device 1212, it should be appreciated by those skilled in the art that computer-readable data storage media can be any available non-transitory, physical device or article of manufacture from which the task assignment server 106 can read data and/or instructions. The computer-readable storage media can be comprised of entirely non-transitory media. The mass storage device 1212 is an example of a computer-readable storage device.

Computer-readable data storage media include volatile and non-volatile, removable, and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid-state memory technology, or any other medium which can be used to store information, and which can be accessed by the device.

The task assignment server 106 operates in a networked environment using logical connections to the devices 102 through the communications network 116. The task assignment server 106 connects to the communications network 116 through a network interface unit 1218 connected to the system bus 1206. The network interface unit 1218 can also connect to additional types of communications networks and devices, including through Bluetooth, Wi-Fi, and cellular telecommunications networks including 4G and 5G networks.

The network interface unit 1218 may also connect the task assignment server 106 to additional networks, systems, and devices such as the ADT system 108 and the RTLS 110 shown in FIG. 1, and to a digital health gateway, electronic medical record (EMR) system, vital signs monitoring devices, and other clinical resource centers.

The task assignment server 106 can also include an input/output unit 1220 for receiving and processing inputs and outputs from a number of peripheral devices. Examples of peripheral devices may include, without limitation, a camera, a touchscreen, speakers, a microphone, and similar devices used for voice and video communications.

The mass storage device 1212 and the RAM 1208 can store software instructions and data. The software instructions can include an operating system 1214 suitable for controlling the operation of the task assignment server 106. The mass storage device 1212 and/or the RAM 1208 can also store software instructions 1216, which when executed by the processing device 1202, cause the device to provide the functionality of the task assignment server 106 discussed herein.

The various embodiments described above are provided by way of illustration only and should not be construed to be limiting in any way. Various modifications can be made to the embodiments described above without departing from the true spirit and scope of the disclosure.

What is claimed is:

1. A system for assigning nursing tasks, the system comprising:
at least one processing device; and
at least one memory storage device storing instructions which, when executed by the at least one processing device, cause the at least one processing device to:
determine a staff shortage in a clinical care environment exists;
categorize tasks based on skill level, the tasks being categorized into a first group of tasks for completion by a first type of personnel, and into a second group of tasks for completion by a second type of personnel, the second type of personnel having a skill set different from that of the first type of personnel; and
route a task of the second group to a member of the second type of personnel.

2. The system of claim 1, wherein the staff shortage is determined by receiving a trigger input from a manager of the first type of personnel.

3. The system of claim 1, wherein the staff shortage is determined when average wait times for completion of the tasks exceed a threshold set for the clinical care environment.

4. The system of claim 1, wherein the staff shortage is determined when a ratio of the first type of personnel to patients exceeds a threshold set for the clinical care environment.

5. The system of claim 4, wherein the ratio of the first type of personnel to patients is calculated by determining a total number of the first type of personnel logged into the system, and by determining a total number of patients admitted to the clinical care environment using data acquired from an admission, discharge, and transfer system.

6. The system of claim 4, wherein the ratio of the first type of personnel to patients is calculated by determining a total number of the first type of personnel in the clinical care environment using location data, and determining a total number of patients admitted to the clinical care environment using data acquired from an admission, discharge, and transfer system.

7. The system of claim 6, wherein the location data is acquired from a real-time locating system.

8. The system of claim 1, wherein the task is routed to the member of the second type of personnel based on proximity of the member to a location for completion of the task.

9. The system of claim 1, wherein the task is routed to the member of the second type of personnel based on at least one of an availability of the member, a skill level of the member, a physical ability of the member, one or more preferences of the member, and a location of the member relative to a patient associated with the task in the clinical care environment.

10. The system of claim 1, wherein the instructions, when executed by the at least one processing device, further cause the at least one processing device to:
provide an option to the member of the second type of personnel to accept or decline the task of the second group; and
when the member of the second type of personnel declines the task, reroute the task to another member of the second type of personnel.

11. The system of claim 1, wherein the instructions, when executed by the at least one processing device, further cause the at least one processing device to:
issue an award to the member of the second type of personnel based on monitoring performance of the tasks by the member of the second type of personnel.

12. The system of claim 1, wherein the first type of personnel includes nursing staff members, and the second type of personnel include volunteers and administrative staff.

13. A method of assigning nursing tasks, the method comprising:
determining a staff shortage in a clinical care environment exists;
categorizing tasks based on skill level, the tasks being categorized into a first group of tasks for completion by a first type of personnel, and into a second group of tasks for completion by a second type of personnel, the second type of personnel having a skill set different from that of the first type of personnel; and
routing a task of the second group to a member of the second type of personnel.

14. The method of claim 13, further comprising:
determining the staff shortage exists by receiving a trigger input from a manager of the first type of personnel.

15. The method of claim 13, further comprising:
determining the staff shortage exists when average wait times for completion of the tasks exceed a threshold set for the clinical care environment.

16. The method of claim 13, further comprising:
determining the staff shortage exists when a ratio of the first type of personnel to patients exceeds a threshold set for the clinical care environment.

17. A non-transitory computer readable storage medium, comprising instructions stored thereon which, when read and executed by one or more computing devices, cause the one or more computing devices to:
determine a staff shortage in a clinical care environment exists;
categorize tasks based on skill level, the tasks being categorized into a first group of tasks for completion by a first type of personnel, and into a second group of tasks for completion by a second type of personnel, the second type of personnel having a skill set different from that of the first type of personnel; and
route a task of the second group to a member of the second type of personnel.

18. The non-transitory computer readable storage medium of claim 17, wherein the staff shortage is determined by receiving a trigger input from a manager of the first type of personnel.

19. The non-transitory computer readable storage medium of claim 17, wherein the staff shortage is determined when average wait times for completion of the tasks exceed a threshold set for the clinical care environment.

20. The non-transitory computer readable storage medium of claim 17, wherein the staff shortage is determined when a ratio of the first type of personnel to patients exceeds a threshold set for the clinical care environment.
